(19) United States
(12) Patent Application Publication
(10) Pub. No.: US 2012/0323077 A1
(43) Pub. Date: Dec. 20, 2012

(54) STEERABLE TUBE, ENDOSCOPIC INSTRUMENT AND ENDOSCOPE COMPRISING SUCH A TUBE, AND AN ASSEMBLY

(75) Inventor: Marcel Antonius Elisabeth VERBEEK, Simpelveld (NL)

(73) Assignee: FORTIMEDIX B.V., Nuth (NL)

(21) Appl. No.: 13/160,949

(22) Filed: Jun. 15, 2011

(51) Int. Cl. A61B 1/00

(52) U.S. Cl. 600/146

(57) ABSTRACT

A steerable tube for endoscopic applications includes a first, a second and a third part. The first part includes a first movable portion and the third part includes a second movable portion. The steerable tube includes longitudinal elements that are constructed and arranged such that a change in orientation of the first movable portion of the first part with respect to the second part results in a change in orientation of the second movable portion of the third part with respect to the second part. The longitudinal elements are constructed and arranged such that they are twisted and/or that they are twistable. An endoscopic instrument and an endoscope including the steerable tube, and an assembly including an endoscopic instrument and the endoscope are also described.

### FIELD OF THE INVENTION

The present invention relates to a steerable tube for endoscopic applications comprising a first, a second and a third part, wherein the first part comprises a first movable portion that is constructed and arranged for movably connecting the first part to a first end of the second part, wherein the third part comprises a second movable portion that is constructed and arranged for movably connecting the third part to a second end of the second part, wherein the steerable tube comprises longitudinal elements that are constructed and arranged such that a change in orientation of the first movable portion of the first part with respect to the second part results in a change in orientation of the second movable portion of the third part with respect to the second part.

Such a steerable tube can be applied in endoscopic instruments and endoscopes. In minimal invasive surgical interventions an assembly of an endoscopic instrument and an endoscope is commonly used for reaching remote operating areas inside a body. If the endoscopic instrument and/or the endoscope of such an assembly comprise a steerable tube, reaching of these areas can be facilitated as steerability of the assembly can be improved. As a result, an operator, e.g. a physician, can more easily guide the assembly through for example intestines, bronchia, joints and other body tissues or cavities towards the operating area without seriously damaging walls of these structures or surrounding tissue. In addition, steerable tubes comprising multiple independent movable portions provide the possibility of making S-like curves. This enables enhanced positioning of a surgical tool of the endoscopic instrument at the operating area.

Endoscopic instruments can also be used for other than medical applications. Examples include inspection and/or repair of mechanical or electronic installations at locations that are difficult to reach. In the following description, terms as endoscopic application or endoscopic instrument might be used. However, these terms must be interpreted as covering also other applications or instruments.

The present invention further relates to an endoscopic instrument comprising a steerable tube according to the invention.

The present invention also relates to an endoscope comprising a steerable tube according to the invention.

The present invention even further relates to an assembly comprising an endoscopic instrument and an endoscope according to the invention.

### BACKGROUND OF THE INVENTION

Transformation of surgical interventions requiring large incisions for exposing an operation area into minimal invasive surgical interventions, i.e. requiring only small incisions for establishing access to the operation area, is a well-known and ongoing trend. To be able to perform a minimal invasive surgical intervention, a physician needs to have access to surgical instruments that enable reaching the operation area via a small incision and to remotely perform actions at the operation area. Endoscopic instruments are well-known in the art and commonly comprise a steerable tube comprising a first, a second and a third part, a handle that is connected to the first part and a tool, e.g. a biopsy cutter, a pair of scissors, pliers, grippers, connected to the third part. In medical applications the first and third parts of the steerable tube are commonly indicated as proximal and distal parts respectively. The terms proximal and distal are defined with respect to the physician that operates the instruments, i.e. the physician manipulates the proximal end of the instrument which results in an event at the distal end of the instrument at the operation area inside a patient's body.

A detailed description of steerable tubes and preferred processes for producing them have been described in international patent applications WO 2009/112060 and WO 2009/127236 of the applicant, which applications were filed on 30.06.2008 and 18.04.2008, respectively and are here incorporated by reference.

Known steerable tubes according to the above mentioned international patent applications of the applicant comprise longitudinal elements that are constructed and arranged such that a change in orientation of the first movable portion of the first or proximal part with respect to the second part results in a change in orientation of the second movable portion of the third or distal part with respect to the second part in an opposite direction.

This steering characteristic of the known steerable tubes is a first drawback for operators that prefer the change in orientation of the third or distal part with respect to the second part of the steerable tube to be in a same direction as the change in orientation of the first or proximal part with respect to the second part because their eye-hand coordination is adjusted to such steering behavior. Although operators, e.g. physicians, can of course learn to adjust their eye-hand coordination in order to steer known steerable tubes of the kind described above, this might not be a realistic option in emergency situations in which there commonly is insufficient time to get adjusted to a different eye-hand coordination. It is clear that a malfunctioning eye-hand coordination is a serious problem that leads to increased risks for both a patient and the instruments used. For example tissues and body parts could be seriously damaged due to collisions with surgical tools located at the third or distal parts of the steerable tubes. The surgical tool themselves could also get damaged due to collisions with each other.

A second drawback of the known steerable tubes with the above mentioned opposite steering characteristic is that it is not possible to achieve an assembly comprising at least two endoscopic instruments and an endoscope, wherein the endoscope has a diameter that is as small as possible. The reason for this is that the use of such known steerable tubes prevents the two endoscopic instruments to be positioned next to each other as closely as possible because the first or proximal parts of the steerable tubes would be in each other's way. This situation is even worse if handles are connected to the first or proximal parts for manipulating the tools located at the third or distal parts of the steerable tubes of the endoscopic instruments.

### SUMMARY OF THE INVENTION

It is an object of the present invention to provide a steerable tube for endoscopic applications that preempts or at least reduces the abovementioned drawbacks of the abovementioned known steerable tubes. It is also an object of the present invention to provide an endoscopic instrument comprising a steerable tube according to the invention. It is a further object of the present invention to provide an endoscope comprising a steerable tube according to the invention. It is an even further object of the present invention to provide an assembly comprising an endoscopic instrument and an endoscope according to the invention.

At least one of these objects is achieved by a steerable tube according to the present invention, wherein the longitudinal elements are constructed and arranged such that they are twisted and/or that they are twistable. By twisting of the longitudinal elements a direction of the change in orientation of the second movable portion of the third part with respect to the second part of the steerable tube as a result of the change in orientation of the first movable portion of the first part with respect to the second part can freely be chosen. The direction of the change is adjustable by selecting an angle of twist of the longitudinal elements. The angle of twist is defined as the angle along the circumference of the steerable tube between a first end of a longitudinal element at a first end of the steerable tube and a second end of the longitudinal element at a second end of the steerable tube. The angle of twist can be any angle out of 0°-360°. If the angle of twist is equal to 180°, the orientation of the second movable portion of the third or distal part as a result of a change in orientation of the first movable portion of the first or proximal part changes in the same direction with respect to the second part of the steerable tube. Other angles of twist, for example 0°, 45°, 60°, 90°, 120°, 135°, 225°, 270°, 315° etc. can be selected for achieving a desired direction of the change in orientation of the second movable portion of the third or distal part with respect to the second part of the steerable tube as a result of the change in orientation of the first movable portion of the first or proximal part with respect to the second part of the steerable tube.

In an embodiment of the steerable tube according to the present invention, the angle of twist of longitudinal elements is preset. In this case, the longitudinal elements have been twisted during manufacturing of the steerable tube and the angle of twist is not adjustable anymore. In an embodiment of the steerable tube all longitudinal elements have the same preset angle of twist. In another embodiment, the steerable tube comprises several separate sets of longitudinal elements of which several sets can have a first preset angle of twist and other sets can have a second preset angle of twist. It will be clear to the person skilled in the art that many combinations are possible without departing from the scope of the present invention.

In an embodiment of the steerable tube according to the present invention, the angle of twist of longitudinal elements is adjustable. In this case, the steerable tube is constructed and arranged such that the operator can adjust the angle of twist such that a desired steering characteristic is obtained. It is possible that the angle of twist of several separate sets of longitudinal elements is preset and that the angles of twist of other separate sets of longitudinal elements are adjustable. It will be clear to the person skilled in the art that many combinations are possible without departing from the scope of the present invention.

In an embodiment of the steerable tube according to the present invention, the steerable tube comprises an inner, an intermediate and an outer concentric layer, wherein the inner and outer layers are constructed and arranged for shielding the intermediate layer, wherein the intermediate layer comprises longitudinal elements, wherein the inner, intermediate and outer layers comprise movable portions in the first and third parts of the steerable tube, wherein movable portions of the first parts of the inner, intermediate and outer layers and movable portions of the third parts of these layers are located opposite to each other in a radial direction of the steerable tube. Such steerable tubes have been described in international patent application WO 2009/112060 of the applicant, which is here incorporated by reference. An effect of the composition of these steerable tubes is a reduction of manufacturing costs as it can be avoided to implement the longitudinal elements for example using cables or wires that need to be connected via connecting elements such as bolts and nuts or soldered joints to the first and third parts of the steerable tube.

In an embodiment of the steerable tube according to the present invention, the intermediate layer comprises longitudinal slits for forming the longitudinal elements. In order to enhance the flexibility to the movable portions of the intermediate layer, the longitudinal slits in those portions of the intermediate layer can be made wider in a circumferential direction of the steerable tube. Such longitudinal slits and elements have been described in international patent applications WO 2009/112060 and WO 2009/127236 of the applicant, which are here incorporated by reference.

In an embodiment of the steerable tube according to the present invention a spacer element is provided between the longitudinal elements. At least one spacer element can for example be provided in the longitudinal slit between neighboring longitudinal elements and/or at least one longitudinal element can for example be provided with at least one spacer element that extends in the longitudinal slit towards a neighboring longitudinal element for keeping the neighboring longitudinal elements at a distance from each other. Such spacer elements have been described in detail in international patent application WO 2009/112060 of the applicant, which is here incorporated by reference.

In an embodiment of the steerable tube according to the present invention, the first and third parts comprise a corresponding number of at least two independent movable portions, wherein changes in orientation of the first and a third independent movable portion of the first part with respect to the second part are transferable to respectively the second and a fourth independent movable portion of the third part via separate sets of longitudinal elements. These independent movable portions located at the first or proximal and third or distal parts of the steerable tube provide for example the possibility of making S-like curves. This capability enables enhanced steerability and/or positioning of a tool, e.g. a surgical tool, that is located at the third or distal part of the steerable tube of an endoscopic instrument. Such steerable tubes have been described in international application WO 2009/127236 of the applicant, which is here incorporated by reference.

In an embodiment of the steerable tube according to the present invention, the first and third parts comprise a corresponding number of at least two independent movable portions, wherein changes in orientation of the first and a third independent movable portion of the first part with respect to the second part are transferable to respectively the second and a fourth independent movable portion of the third part via separate sets of longitudinal elements, wherein the intermediate layer comprises at least two concentric layers, wherein each concentric layer is constructed and arranged for comprising at least one separate set of longitudinal elements. In the case that several sets of longitudinal elements are arranged in the same concentric layer of the intermediate layer, the angles of twist of these separate sets are not adjustable independently. However, an advantage of this embodiment is that the diameter of the steerable tube can be minimized as the intermediate layer does not comprise a separate concentric layer for each separate set of longitudinal elements. Such steerable tubes have been described in international patent application WO 2009/127236 of the applicant, which is here incorporated by reference.

In an embodiment of the steerable tube according to the present invention, each separate set of longitudinal elements is arranged in a separate concentric layer of the intermediate layer. In this case, the angle of twist of each separate set of longitudinal elements can be adjusted independently. This enhances the degree of freedom with respect to adjusting the steering characteristic of the steerable tube, i.e. which the direction of the change in orientation of the movable portions of the third or distal part is with respect to the second part as a result of a change in orientation of the corresponding movable portions of the first or proximal part with respect to the second part. However, the enhanced degree of freedom with respect to adjusting the steering characteristic comes at the cost of a larger diameter of the steerable tube as the intermediate layer comprises separate concentric layers for accommodating each separate set of longitudinal elements.

In an embodiment of the steerable tube according to the present invention, the steerable tube comprises an adjustment device that is constructed and arranged for adjusting the angle of twist of longitudinal elements. The adjustment device is for example a wheel, a knob, a ball or a lever, that is connected to the first or proximal part of the steerable tube and is constructed such that the angle of twist for example is adjustable per degree. In order to adjust the angles of twist of several separate sets of longitudinal elements, several adjustment devices, for example one for each separate set, can be applied. The adjustment device could also be part of a handle that is connectable to the first or proximal part of the steerable tube. It will be clear to the person skilled in the art that many configurations of at least one adjustment device can be envisaged, which configurations all fall within the scope of the present invention.

In an embodiment of the steerable tube according to the present invention, the adjustment device is connectable to the intermediate layer. In an exemplary embodiment, a steerable tube comprises an inner and an outer layer having a first, a second and a third part, wherein both the first and third parts of both the inner and outer layers comprise only one movable portion. In this case, the steerable tube further comprises an intermediate layer, which is arranged between the inner and outer layers, having only one concentric layer for accommodating the longitudinal elements that are constructed and arranged for transferring a change in orientation of the movable portions of the first parts of the inner and outer layers with respect to the second part to the movable portions of the third parts of the inner and outer layers. The adjustment device is for example connectable to a first end of the intermediate layer that is located at the first or proximal part of the steerable tube. A second end of the intermediate layer is arranged at the third or distal part of the steerable tube such that the longitudinal elements are twistable by displacing the adjustment device, e.g. by rotating the adjustment device in a circumferential direction of the steerable tube.

In an exemplary embodiment both the first and third parts of both the inner and outer layers comprise for example three independent movable portions. If in this case, the intermediate layer comprises three concentric layers, i.e. one for each separate set of longitudinal elements, three adjustment devices are required each of which is connected for example to a first end of each separate concentric layer of the intermediate layer that is arranged at the first or proximal part of the steerable tube for independently adjusting the angles of twist of each separate set of longitudinal elements. It will be clear to the person skilled in the art that many configurations for connecting at least one adjustment device to at least one concentric layer of the intermediate layer can be envisaged, which configurations all fall within the scope of the present invention.

In an embodiment of the steerable tube according to the present invention, the adjustment device is connectable to the inner and/or the outer layer. If the adjustment device is connected to the inner layer, the inner and intermediate layers have to be arranged such that a displacement of the inner layer as a result of a displacement of the adjustment device, e.g. in a circumferential direction of the steerable tube, is transferrable to the intermediate layer such that the angle of twist of the longitudinal elements is adjustable. The same applies to an embodiment wherein the adjustment device is connected to the outer layer. It would also be possible that an adjustment device is connectable to both the inner and the outer layer. It will be clear to the person skilled in the art that many configurations for connecting an adjustment device to the inner and/or the outer layer can be envisaged without departing from the scope of the present invention.

In an embodiment of the steerable tube according to the present invention, the steerable tube comprises a locking device that is constructed and arranged for maintaining a position of the adjustment device when a desired angle of twist has been selected. The locking device can comprise a locking bolt or screw, a ratchet construction, a friction construction or a double-throw or multiple-throw construction. It will be clear to the person skilled in the art that many configurations of the locking mechanism can be envisaged without departing from the scope of the present invention.

In an embodiment of the steerable tube according to the present invention, the steerable tube comprises an inner, an intermediate and an outer concentric layer, wherein the inner and outer layers are constructed and arranged for shielding the intermediate layer, wherein the intermediate layer comprises longitudinal elements, wherein the inner, intermediate and outer layers comprise movable portions in the first and third parts of the steerable tube, wherein movable portions of the first parts of the inner, intermediate and outer layers and movable portions of the third parts of these layers are located opposite to each other in a radial direction of the steerable tube, wherein the steerable tube comprises a compensating device that is constructed and arranged for compensating a change in length of the intermediate layer in a longitudinal direction of the steerable tube upon twisting and/or untwisting the longitudinal elements. The length of the intermediate layer in the longitudinal direction of the steerable tube decreases due to twisting the longitudinal elements. Upon untwisting the longitudinal elements the length of the intermediate layer in the longitudinal direction of the steerable tube increases. Compensation of the change in length of the intermediate layer can be achieved because the movable portions of the first and/or the third parts for example comprise a compensating device according to the present invention. The compensating device provides the movable portions of the first and/or the third parts with sufficient flexibility in the longitudinal direction of the steerable tube for compensating changes in the length of the intermediate layer. It will be clear to the person skilled in the art that compensating devices providing sufficient flexibility to the movable portions for compensating the change in length of the intermediate layer due to twisting and/or untwisting the longitudinal elements can be constructed and arranged in many ways without departing from the scope of the present invention.

In an embodiment of the steerable tube according to the present invention, the steerable tube comprises a compensating device that is constructed and arranged for prestressing the longitudinal elements at least when the angle of twist is substantially zero degrees (0°). If the flexibility of the movable portions of the intermediate layer in the longitudinal direction of the steerable tube is insufficient, an additional compensating device can for example be applied that is constructed and arranged for compensating the change in length of the intermediate layer in the longitudinal direction of the steerable tube upon twisting and/or untwisting the longitudinal elements. The compensating device can for example comprise a resilient element, e.g. a spring and/or a rubber tube, that prestresses the longitudinal elements at least when the angle of twist is substantially zero degrees, i.e. when they are untwisted.

In an embodiment of the steerable tube according to the present invention, the adjustment device can be constructed and arranged to comprise a compensating device for compensating a change in length of the intermediate layer in a longitudinal direction of the steerable tube upon twisting and/or untwisting the longitudinal elements. The adjustment device can for example be constructed and arranged for being movable along a helical line that is constructed and arranged on the steerable tube. The helical line comprises an angle of twist that is constructed and arranged such that when the adjustment device is displaced over a certain angle of rotation, a distance that the adjustment device travels along the helical line is equal to the change in length of the intermediate layer in the longitudinal direction of the steerable tube. It will be clear to the person skilled in the art that many configurations of the adjustment device can be envisaged without departing from the scope of the present invention.

According to another aspect of the present invention, an endoscopic instrument is provided comprising a steerable tube according to the invention. The endoscopic instrument for example further comprises a tool that is constructed and arranged at the third or distal part of the steerable tube and a handle that is constructed and arranged at the first or proximal part of the steerable tube for steering the endoscopic instrument and controlling the tool. An example of such a handle is described in U.S. patent application Ser. No. 13/069,662 of the applicant and is here incorporated by reference. The steerability of the endoscopic instrument is significantly improved as the steering characteristic of the steerable tube according to the present invention can be adjusted to the desire of the operator. Due to an improved steering and/or positioning capability of the endoscopic instrument comprising a steerable tube according to the present invention, the operator will be able to perform more complicated actions. In addition, the operator will feel more comfortable when performing critical manipulations as he can have significantly improved control over the endoscopic instrument. This contributes to a reduction of the risks involved in critical operations.

According to another aspect of the present invention an endoscope is provided comprising a steerable tube according to the present invention. The endoscope for example further comprises a handle that is constructed and arranged at a first or proximal part of the steerable tube. The steerability of the endoscope is significantly improved as the steering characteristic of the steerable tube according to the present invention can be adjusted to the desire of the operator. As a result, the operator, e.g. a physician, can more easily guide the endoscope through for example intestines, bronchia, joints and other body tissues or cavities towards the operating area without seriously damaging walls of these structures or surrounding tissue.

According to another aspect of the present invention an assembly is provided comprising an endoscopic instrument and an endoscope according to the present invention. An exemplary assembly according to the present invention comprises two endoscopic instruments that are arranged in the steerable tube of an endoscope. For such an assembly the diameter of the steerable tube of the endoscope can be reduced as much as possible as the endoscopic instruments can be arranged next to each other as closely as possible by selecting the steering characteristics of their steerable tubes such that the tools at the third or distal parts of the steerable tubes are not in each other's way when in use. Due to an improved steering and/or positioning capability of the endoscopic instruments and/or endoscope of an assembly according to the present invention, the operator will be able to perform more complicated actions.

### BRIEF DESCRIPTION OF THE DRAWINGS

These and other features and effects of the present invention will be explained in more detail below with reference to drawings in which preferred and illustrative embodiments of the invention are shown. The person skilled in the art will realize that other alternatives and equivalent embodiments of the invention can be conceived and reduced to practice without departing from the scope of the present invention.

FIG. 1a shows a schematic perspective view of an embodiment of a steerable tube according to the present invention.

FIG. 1b shows a schematic exploded view of an embodiment of a steerable tube according to the present invention, wherein an angle of twist of the longitudinal elements is zero degrees (0°), i.e. the longitudinal elements are untwisted.

FIG. 1c shows a schematic view of the steerable tube of FIG. 1b, wherein a change in orientation of a second movable portion of a third part with respect to a second part of the steerable tube is in a direction opposite to the direction of the change in orientation of a first movable portion of a first part with respect to the second part.

FIG. 1d shows a schematic exploded view of an embodiment of a steerable tube according to the present invention, wherein the angle of twist of the longitudinal elements is preset to 180 degrees (180°).

FIG. 1e, shows a schematic view of the steerable tube of FIG. 1d, wherein the direction of the change in orientation of the second movable portion of the third part with respect to the second part of the steerable tube is the same as the direction of the change in orientation of the first movable portion of the first part with respect to the second part.

FIG. 2a shows a schematic perspective view of an embodiment of a steerable tube according to the present invention comprising an adjustment device having one wheel that is constructed and arranged at the first part of the steerable tube for adjusting the angle of twist of the longitudinal elements. The wheel of the adjustment device is in a first position in which the angle of twist is 0°, i.e. the longitudinal elements are untwisted.

FIG. 2b shows a schematic perspective view of the steerable tube, wherein the wheel of the adjustment device is in a second position in which the angle of twist is 180°, i.e. the longitudinal elements are twisted.

FIG. 2c shows a schematic perspective view of an embodiment of a steerable tube according to the present invention, wherein the steerable tube comprises a first and a third part both comprising two independently movable portions. The steerable tube further comprises an adjustment device having two wheels that are constructed and arranged for independently adjusting the angles of twist of the two separate sets of longitudinal elements that are constructed and arranged in two concentric layers of an intermediate layer of the steerable tube.

FIG. 3a shows a combination of a schematic side view and a longitudinal cross sectional view of a part of an embodiment of a steerable tube according to the present invention, wherein the steerable tube comprises a spring as a compensating mechanism that is constructed and arranged for prestressing the longitudinal elements at least when the angle of twist is substantially equal to 0° and a friction element that serves as a locking device for maintaining the adjustment device in a position after the desired angle of twist has been selected.

FIG. 3b shows a combination of a schematic side view and a longitudinal cross sectional view of the part of the embodiment of the steerable tube shown in FIG. 3a. The spring is compressed due to shortening of the intermediate layer as a result of twisting of the longitudinal elements.

FIG. 4a shows a schematic side view of an embodiment of a steerable tube according to the present invention, wherein the outer layer of the steerable tube has partially been opened for showing the longitudinal elements. The steerable tube shown in FIG. 4a comprises an operating member that is constructed and arranged for being movable along a helical line that is constructed and arranged on the steerable tube. The helical line comprises an angle of twist that is constructed and arranged such that when the operating member is displaced over a certain angle of rotation, a distance that the adjustment device travels along the helical line is equal to the change in length of the intermediate layer in the longitudinal direction of the steerable tube. The operating member as shown in FIG. 4a is in a first position in which the angle of twist of the longitudinal elements is 0°, i.e. the longitudinal elements are untwisted.

FIG. 4b shows a schematic side view of the embodiment of the steerable tube shown in FIG. 4a. The operating member as shown in FIG. 4b is in a second position in which the angle of twist of the longitudinal elements is for example 180°, i.e. the longitudinal elements are twisted.

FIG. 4c shows a schematic side view of an embodiment of a steerable tube according to the present invention comprising an operating member that is constructed and arranged for being displaceable along a stepped slot that enables maintaining the position of the operating member when a desired angle of twist has been selected. The stepped slot is constructed and arranged such that when the operating member is displaced over a certain distance along the circumference of the steerable tube, the change in length of the intermediate layer in the longitudinal direction of the steerable tube is compensated. The operating member as shown in FIG. 4c is in a first position in which the angle of twist of the longitudinal elements is 0°, i.e. the longitudinal elements are untwisted.

FIG. 4d shows a schematic side view of the embodiment of the steerable tube shown in FIG. 4c. The operating member as shown in FIG. 4b is in a second position in which the angle of twist of the longitudinal elements is for example 180°, i.e. the longitudinal elements are twisted.

FIG. 5 shows a combination of a schematic side view and a longitudinal cross sectional view of an assembly according to the present invention comprising two endoscopic instruments that are arranged next to each other as closely as possible in the tube of an endoscope.

The figures are not necessarily drawn to scale. In the figures identical components are denoted by the same reference numerals.

### DETAILED DESCRIPTION

The steerable tube as shown in the annexed drawings can be applied in endoscopic instruments and endoscopes for medical applications but its use is not restricted to that. It may also be used in other applications such as technical applications in which endoscopic instruments and/or endoscopes are used for handling or viewing parts of machines or installations which are otherwise difficult to reach. The steerable tube according to the present invention as used in this description will implicitly include these applications.

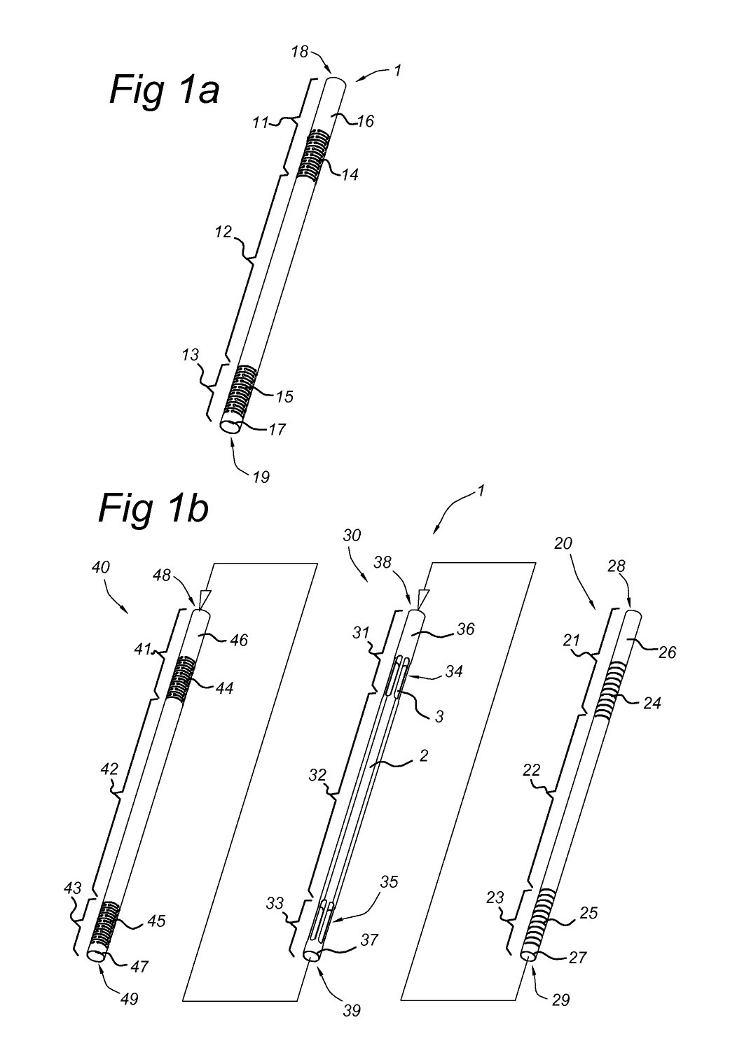

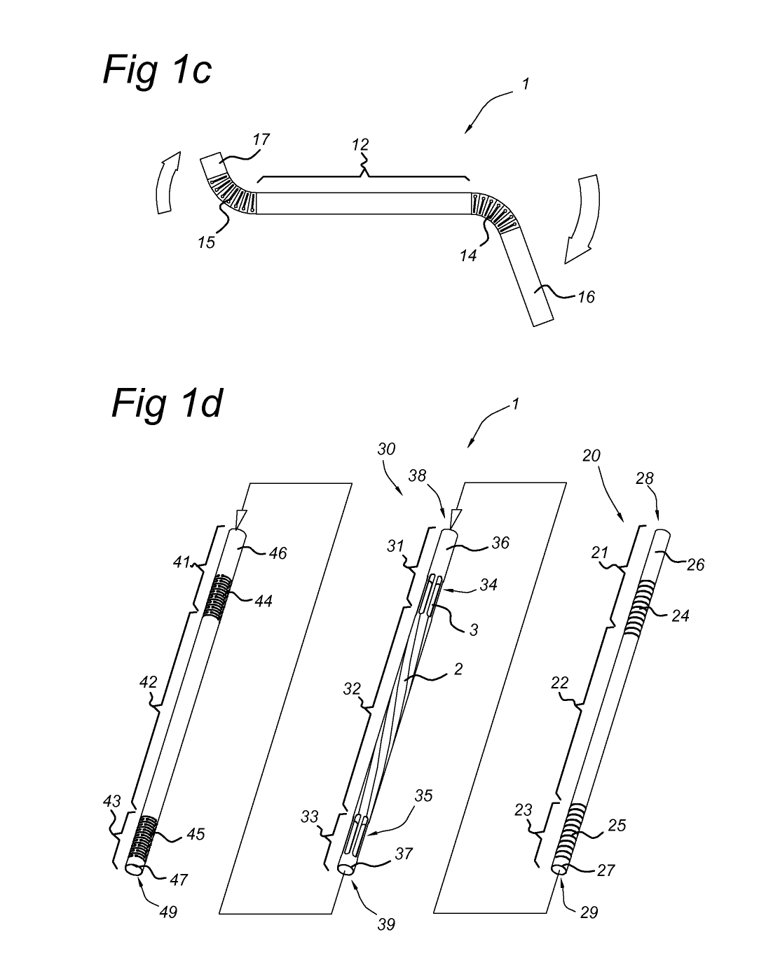

FIG. 1a shows a schematic perspective view of an embodiment of a steerable tube 1 according to the present invention comprising a first 11, a second 12 and a third 13 part. The first part 11 comprises one movable portion 14 with which it is connected to a first end of the second part 12. The first part 11 further comprises one rigid portion 16 that forms a first end 18 of the steerable tube 1. The third part 13 comprises one movable portion 15 with which it is connected to a second end of the second part 12. The third part 13 further comprises one rigid portion 17 that forms a second end 19 of the steerable tube 1.

FIG. 1b shows a schematic exploded view of an embodiment of a steerable tube 1 according to the present invention, wherein an angle of twist of the longitudinal elements 2 is 0°, i.e. the longitudinal elements 2 are untwisted. The steerable tube 1 comprises an inner 20, an intermediate 30 and an outer 40 concentric layer. Each of these layers comprises a first 21, 31, 41, a second 22, 32, 42, and a third 23, 33, 43 part. Each of the inner 20, intermediate 30 and outer 40 layers comprises one movable portion 24, 34, 44 in their first 21, 31, 41 parts and one movable portion 25, 35, 45 in their third 23, 33, 43 parts, wherein the movable portions 24, 34, 44 of the first parts 21, 31, 41 of the inner 20, intermediate 30 and outer 40 layers and the movable portions 25, 35, 45 of the third parts 23, 33, 43 of these layers are constructed and arranged for being located opposite to each other in a radial direction of the steerable tube 1.

The inner 20 and outer 40 layers are constructed and arranged for shielding the intermediate layer 30 that comprises longitudinal elements 2 that are constructed and arranged such that a change in orientation of the first part 11 of the steerable tube 1 with respect to the second part 12 results in a change in orientation of the third part 13 with respect to the second part 12. The steering characteristic of the steerable tube 1, i.e. which the direction of the change in orientation of the movable portion 15 of the third or distal part 13 is with respect to the second part 12 as a result of a change in orientation of the corresponding movable portion 14 of the first or proximal part 11 with respect to the second part 12, depends on the angle of twist of the longitudinal elements 2. The longitudinal elements 2 comprise longitudinal parts of the intermediate layer 30 and are separated by longitudinal slits 3 that are wider in a circumferential direction of the steerable tube 1 in the movable portions 34, 35 of the first 31 and third 33 parts of the intermediate layer 30.

In a different embodiment of the steerable tube 1 according to the present invention a spacer element can be provided between the longitudinal elements 2. At least one spacer element can for example be provided in the longitudinal slit 3 between neighboring longitudinal elements 2 and/or at least one longitudinal element 2 can for example be provided with at least one spacer element that extends in the longitudinal slit 3 towards a neighboring longitudinal element 2 for keeping the neighboring longitudinal elements 2 at a distance from each other. Details with respect to such spacer elements have been described in detail in international patent application WO 2009/112060 of the applicant, which is here incorporated by reference.

The first parts 21, 31, 41 of the inner 20, intermediate 30 and outer 40 layers each comprise one rigid portion 26, 36, 46 which forms a first end 28, 38, 48 of each of these layers. The third parts 23, 33, 43 of the inner 20, intermediate 30 and outer 40 layers each comprise one rigid portion 27, 37, 47 which forms a second end 29, 39, 49 of each of these layers. Such steerable tubes 1 have been described in international patent application WO 2009/112060 of the applicant, which is here incorporated by reference.

FIG. 1c shows a schematic view of the steerable tube 1 of FIG. 1b. A change in orientation of the movable portion 15 of the third part 13 with respect to the second part 12 of the steerable tube 1 is in a direction opposite to the direction of the change in orientation of the movable portion 14 of the first part 11 with respect to the second part 12 because the angle of twist of the longitudinal elements 2 is 0°, i.e. the longitudinal elements 2 are untwisted.

FIG. 1d shows a schematic exploded view of an embodiment of a steerable tube 1 according to the present invention, wherein the angle of twist of the longitudinal elements 2 is preset to 180°. In this exemplary embodiment, the longitudinal elements 2 have been constructed and arranged such that only the parts of the longitudinal elements 2 in the second part 12 of the steerable tube 1 are twistable. It will be clear to the person skilled in the art that many configurations can be envisaged for twisting the longitudinal elements 2 without departing from the scope of the present invention. FIG. 1e, shows a schematic view of the steerable tube 1 of FIG. 1d. The direction of the change in orientation of movable portion 15 of the third part 13 with respect to the second part 12 of the steerable tube 1 is the same as the direction of the change in orientation of movable portion 14 of the first part 11 with respect to the second part 12 because the angle of twist of the longitudinal elements 2 is 180°, i.e. the longitudinal elements 2 are twisted as is shown in FIG. 1d.

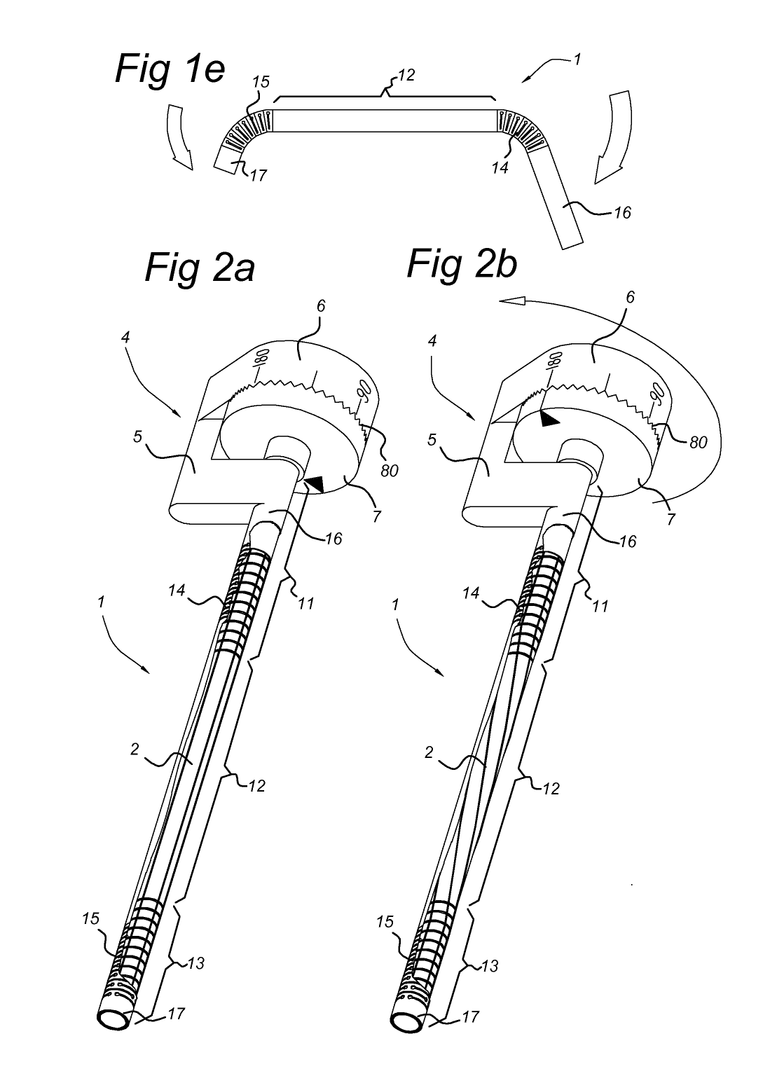

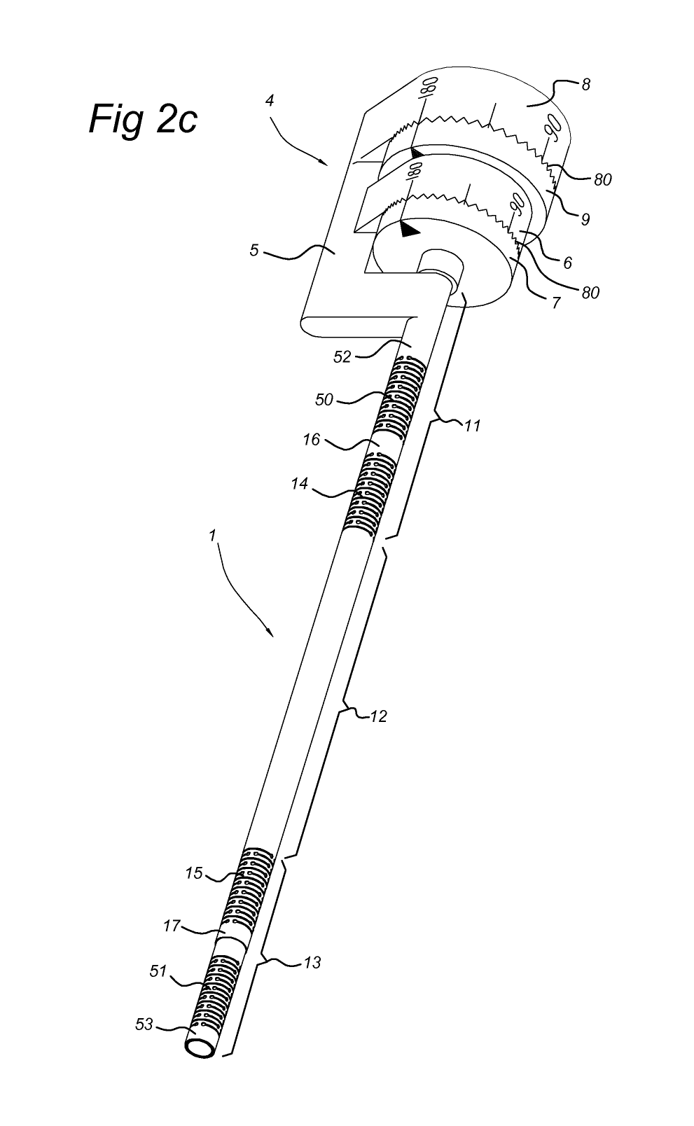

FIG. 2a shows a schematic perspective view of an embodiment of a steerable tube 1 according to the present invention comprising an adjustment device 4 comprising one wheel 7 that is constructed and arranged at the rigid portion 16 of the first part 11 of the steerable tube 1 for adjusting the angle of twist of the longitudinal elements 2. The wheel 7 of the adjustment device 4 is in a first position in which the angle of twist is 0°, i.e. the longitudinal elements 2 of the intermediate layer 30 are untwisted as can be seen through the partial cut away in the outer layer 40.

In the exemplary embodiment of the adjustment device 4 shown in FIG. 2a, the adjustment device 4 comprises a frame 5 that is connected to the rigid portion 16 of the first part 11 of the steerable tube 1. The adjustment device 4 further comprises a part 6 that is provided with a graded arc and the wheel 7 that is constructed and arranged for selecting the angle of twist of the longitudinal elements 2. The wheel 7 comprises a ratchet construction 80 that serves as a locking element for maintaining the wheel 7 in a position after having selected a desired angle of twist. It will be clear to the person skilled in the art that many configurations of the adjustment device 4 can be envisaged without departing from the scope of the present invention.

In an embodiment of the steerable tube 1, the wheel 7 of the adjustment device 4 is for example connectable to the rigid portion 36 of the first part 31 of the intermediate layer 30. The intermediate layer 30 is arranged at the third part 13 of the steerable tube 1 such that the longitudinal elements 2 are twistable by rotating the wheel 7 of the adjustment device 4 in a circumferential direction of the steerable tube 1 indicated by the bend arrow in FIG. 2b.

In an embodiment of the steerable tube 1, the wheel 7 of the adjustment device 4 is connectable to the inner 20 and/or the outer 40 layer. If the wheel 7 is connected to the inner layer 20, the inner 20 and intermediate 30 layers have to be arranged such that a displacement of the inner layer 20 as a result of a rotation of wheel 7 in the circumferential direction of the steerable tube 1, is transferrable to the intermediate layer 30 such that the angle of twist of the longitudinal elements 2 is adjustable. The same applies to an embodiment wherein the wheel 7 is connected to the outer layer 40. It would also be possible that an adjustment device 4 is connectable to both the inner 20 and the outer 40 layer. It will be clear to the person skilled in the art that many configurations for connecting an adjustment device 4 to the inner 20 and/or the outer 40 layer can be envisaged without departing from the scope of the present invention.

FIG. 2b shows a schematic perspective view of the steerable tube 1, wherein the wheel 7 of the adjustment device 4 is in a second position in which the angle of twist of the longitudinal elements 2 has been selected to be 180°, i.e. the longitudinal elements 2 are twisted such that the direction of the change in orientation of the third part 13 of the steerable tube 1 with respect to the second part 12 is the same as the direction of the change in orientation of the first part 11 of the steerable tube with respect to the second part 12.

FIG. 2c shows a schematic perspective view of an embodiment of a steerable tube 1 according to the present invention, wherein the steerable tube 1 comprises a first part 11 having a first 14 and a third 50 independently movable portion and a first 16 and a third 52 rigid portion. The steerable tube 1 further comprises a second part 12 and a third part 13 comprising a second 15 and a fourth 51 independently movable portion and a second 17 and a fourth rigid 53 portion. An adjustment device 4 comprising a first 7 and a second 9 wheel is connected to the third rigid portion 52 of the first part 11 of the steerable tube 1. The wheels 7, 9 are constructed and arranged for independently selecting the angles of twist of the two separate sets of longitudinal elements that are constructed and arranged in two concentric layers of the intermediate layer 30 of the steerable tube 1. The wheels 7, 9 comprise ratchet constructions 80 that serve as locking elements for maintaining the wheels 7, 9 in their positions after having selected the desired angles of twist.

In this exemplary embodiment, the first wheel 7 of the adjustment device 4 is constructed and arranged for adjusting the angle of twist of a first separate set of longitudinal elements that transfers a displacement of the first movable portion 14 of the first part 11 of the steerable tube 1 with respect to the second part 12 to the second movable portion 15 of the third part 13 of the steerable tube 1. As a result, the second wheel 9 is constructed and arranged for adjusting the angle of twist of a second separate set of longitudinal elements that transfers a displacement of the third movable portion 50 of the first part 11 of the steerable tube 1 with respect to the second part 12 to the fourth movable portion 51 of the third part 13 of the steerable tube 1. Details about steerable tubes comprising separate sets of longitudinal elements in the same or different concentric layers of the intermediate layer have been described in detail in international patent application WO 2009/127236 of the applicant, which is here incorporated by reference.

FIG. 2c shows that the angles of twist for both separate sets of longitudinal elements have been selected to be 180°. As a result, the directions of the changes in orientation of the second 15 and fourth 51 movable portions with respect to the second part 12 are the same as the directions of the changes in orientation of the first 14 and third 50 movable portions respectively, with respect to the second part 12.

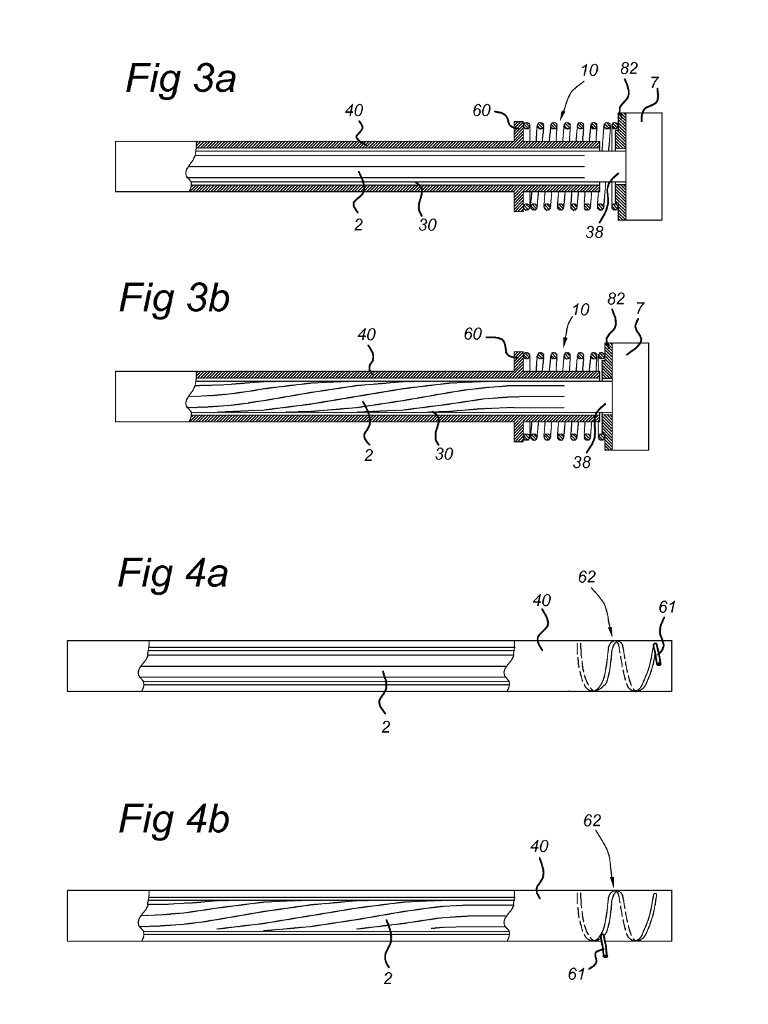

FIG. 3a shows a combination of a schematic side view and a longitudinal cross sectional view of a part of an embodiment of a steerable tube 1 according to the present invention, wherein the steerable tube 1 comprises a spring 10 as a compensating mechanism that is constructed and arranged for prestressing the longitudinal elements 2 at least when the angle of twist is substantially 0°. In the part of the steerable tube 1 shown in FIG. 3a, the spring 10 is arranged between a friction element 82 and a part 60 of the outer layer 40. The friction element 82 serves as a locking device for maintaining the wheel 7 of the adjustment device 4 in a position after selecting a desired angle of twist. It will be clear to the person skilled in the art that many configurations for the compensating mechanism 10 and the friction element 82 can be envisaged without departing from the scope of the present invention.

In the embodiment of the steerable tube 1 shown in FIG. 3a, the adjustment device 4 is connected to a first end 38 of the intermediate layer 30 that is located at the first or proximal part 18 of the steerable tube 1. A second end 39 of the intermediate layer 30 is arranged at the third or distal part 19 of the steerable tube 1 such that the longitudinal elements 2 are twistable by rotating the wheel 7 in a circumferential direction of the steerable tube 1.

FIG. 3b shows a combination of a schematic side view and a longitudinal cross sectional view of the part of the embodiment of the steerable tube 1 shown in FIG. 3a. The spring 10 is compressed due to shortening of the intermediate layer 30 as a result of twisting of the longitudinal elements 2.

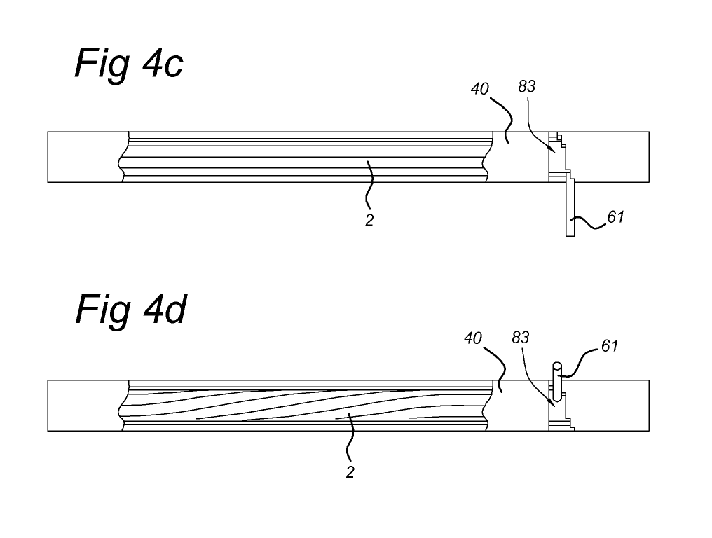

FIG. 4a shows a schematic side view of an embodiment of a steerable tube 1 according to the present invention, wherein the outer layer 40 of the steerable tube 1 has partially been opened for showing the longitudinal elements 2. The steerable tube 1 shown in FIG. 4a comprises an adjustment device that is constructed and arranged as an operating member 61 that is movable along a helical line 62 that in this exemplary embodiment is constructed and arranged in the outer layer 40 of the steerable tube 1. The helical line 62 comprises an angle of twist that is constructed and arranged such that when the operating member 61 is displaced over a certain angle of rotation, a distance that the operating member 61 travels along the helical line 62 is equal to the change in length of the intermediate layer 30 in the longitudinal direction of the steerable tube 1. The operating member 61 as shown in FIG. 4a is in a first position in which the angle of twist of the longitudinal elements 2 is 0°, i.e. the longitudinal elements 2 are untwisted. As a result, the direction of the change in orientation of a movable portion of the third part 13 with respect to the second part 12 is opposite to the direction of the change in orientation of a movable portion of the first part 11 of the steerable tube 1 with respect to the second part 12. It will be clear to the person skilled in the art that many configurations for an adjustment device that is constructed and arranged for compensating a change in length of the intermediate layer 30 of the steerable tube 1 due to twisting and/or untwisting of longitudinal elements 2 can be envisaged without departing from the scope of the present invention.

FIG. 4b shows a schematic side view of the embodiment of the steerable tube 1 shown in FIG. 4a. The operating member 61 as shown in FIG. 4b is in a second position in which the selected angle of twist of the longitudinal elements 2 is for example 180°, i.e. the longitudinal elements 2 are twisted such that the direction of the change in orientation of a movable portion of the third part 13 with respect to the second part 12 is the same as the direction of the change in orientation of a movable portion of the first part 11 of the steerable tube 1 with respect to the second part 12.

FIG. 4c shows a schematic side view of an embodiment of a steerable tube 1 according to the present invention comprising an operating member 61 that is constructed and arranged for being displaceable along a stepped slot 83. The stepped slot 83 serves as a locking device that enables maintaining the position of the operating member 61 when a desired angle of twist has been selected. The stepped slot 83 is constructed and arranged such that when the operating member 61 is displaced over a certain distance along the circumference of the steerable tube 1, the change in length of the intermediate layer 30 in the longitudinal direction of the steerable tube 1 is compensated. The operating member 61 as shown in FIG. 4c is in a first position in which the angle of twist of the longitudinal elements 2 is 0°, i.e. the longitudinal elements 2 are untwisted. It will be clear to the person skilled in the art that many configurations for the operating member 61 and stepped slot 83 can be envisaged without departing from the scope of the present invention.

FIG. 4d shows a schematic side view of the embodiment of the steerable tube 1 shown in FIG. 4c. The operating member 61 as shown in FIG. 4b is in a second position in which the angle of twist of the longitudinal elements 2 is selected to be for example 180°, i.e. the longitudinal elements 2 are twisted.

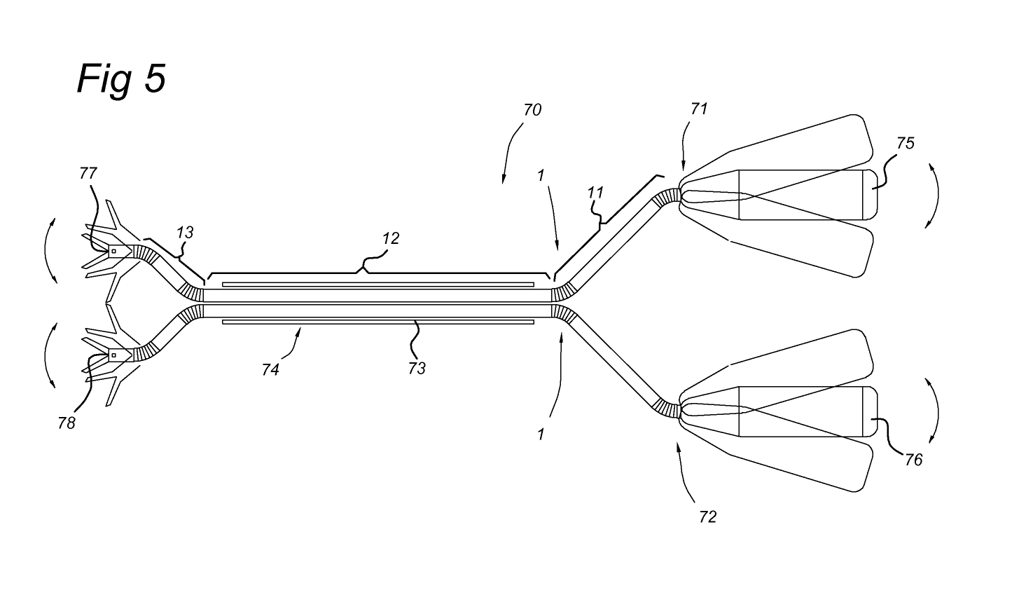

FIG. 5 shows a combination of a schematic side view and a longitudinal cross sectional view of an assembly 70 according to the present invention comprising two endoscopic instruments 71, 72 that are arranged next to each other in the tube 73 of an endoscope 74. In the embodiment of the assembly 70 shown in FIG. 5 the tube 73 preferably is non-steerable. However, if an application so requires, tube 73 can also be steerable. The endoscopic instruments 71, 72 comprise handles 75, 76 that are constructed and arranged at the first or proximal parts 11 of the steerable tubes 1 for steering the endoscopic instruments 71, 72 and controlling the tools 77, 78 that are arranged at the third or distal parts 13 of the steerable tubes 1 of the endoscopic instruments 71, 72. An example of such a handle 75, 76 is described in U.S. patent application Ser. No. 13/069,662 of the applicant and is here incorporated by reference.

Adjustment devices for adjusting the angle of twist of the longitudinal elements can be arranged between the handles 75, 76 and the steerable tubes 1 of the endoscopic instruments 71, 72. It is also possible that the adjustment devices are integrated in the steerable tube 1 and/or in the handles 75, 76. It will be obvious to the person skilled in the art that many configurations for incorporating an adjustment device into the endoscopic instrument without departing from the scope of the present invention.

From FIG. 5 it can clearly be seen that the diameter of the steerable tube 73 of the endoscope 74 can be reduced as much as possible as the endoscopic instruments 71, 72 can be arranged next to each other as closely as possible by selecting the steering characteristics of the steerable tubes 1 such that the tools 77, 78 are not in each other's way when in use. For the exemplary embodiment shown in FIG. 5 the steering characteristics are selected such that the direction of the change in orientation of the movable parts of the third or distal part 13 of each endoscopic instrument 71, 72 with respect to the second part 12 of the steerable tube 1 is the same as the direction of the change of orientation of the movable parts of the first or proximal part 11 of the steerable tube 1 of each endoscopic instrument 71, 72 with respect to the second part 12 of the steerable tube 1. This means that the angle of twist of the longitudinal elements is equal to 180°.

For all illustrative embodiments of the steerable tube 1 according to the present invention shown in the figures described above, it is clear to the person skilled in the art that at least the second parts 22, 32, 42 of the steerable tube 1 can be constructed and arranged for having a bending stiffness, i.e. is more or less rigid, that is suitable for the application envisaged. For some applications the bending stiffness required has to be such that the steerable tube bends as soon as it is picked up from a supporting surface, e.g. a table. For other applications it can be required that this does not happen or at least to a lesser extent.

The present invention can be summarized by the following clauses:

Clause 1. Steerable tube for endoscopic applications comprising a first, a second and a third part, wherein the first part comprises a first movable portion that is constructed and arranged for movably connecting the first part to a first end of the second part, wherein the third part comprises a second movable portion that is constructed and arranged for movably connecting the third part to a second end of the second part, wherein the steerable tube comprises longitudinal elements that are constructed and arranged such that a change in orientation of the first movable portion of the first part with respect to the second part results in a change in orientation of the second movable portion of the third part with respect to the second part, wherein the longitudinal elements are constructed and arranged such that they are twisted and/or that they are twistable.

Clause 2. Steerable tube according to clause 1, wherein a direction of the change in orientation of the second movable portion of the third part with respect to the second part is adjustable by selecting an angle of twist of the longitudinal elements.

Clause 3. Steerable tube according to clause 2, wherein the angle of twist of longitudinal elements is preset.

Clause 4. Steerable tube according to clause 2, wherein the angle of twist of longitudinal elements is adjustable.

Clause 5. Steerable tube according to any one of the preceding clauses, wherein the steerable tube comprises an inner, an intermediate and an outer concentric layer, wherein the inner and outer layers are constructed and arranged for shielding the intermediate layer, wherein the intermediate layer comprises longitudinal elements, wherein the inner, intermediate and outer layers comprise movable portions in the first and third parts of the steerable tube, wherein movable portions of the first parts of the inner, intermediate and outer layers and movable portions of the third parts of these layers are located opposite to each other in a radial direction of the steerable tube.

Clause 6. Steerable tube according to clause 5, wherein the intermediate layer comprises longitudinal slits for forming the longitudinal elements.

Clause 7. Steerable tube according to any one of the preceding clauses, wherein a spacer element is provided between the longitudinal elements.

Clause 8. Steerable tube according to any one of the preceding clauses, wherein the first and third parts comprise a corresponding number of at least two independent movable portions, wherein changes in orientation of the first and a third independent movable portion of the first part with respect to the second part are transferable to respectively the second and a fourth independent movable portion of the third part via separate sets of longitudinal elements.

Clause 9. Steerable tube according to clause 5 and 8, wherein the intermediate layer comprises at least two concentric layers, wherein each concentric layer is constructed and arranged for comprising at least one separate set of longitudinal elements.

Clause 10. Steerable tube according to clause 9, wherein each separate set of longitudinal elements is arranged in a separate concentric layer of the intermediate layer.

Clause 11. Steerable tube according to clause 4, comprising an adjustment device that is constructed and arranged for adjusting the angle of twist of longitudinal elements.

Clause 12. Steerable tube according to clause 5 and 11, wherein the adjustment device is connectable to the intermediate layer.

Clause 13. Steerable tube according to clause 5 and 11, wherein the adjustment device is connectable to the inner and/or the outer layer.

Clause 14. Steerable tube according to clause 11, comprising a locking device that is constructed and arranged for maintaining a position of the adjustment device.

Clause 15. Steerable tube according to any one of the clauses 11-14, wherein the adjustment device comprises the locking device.

Clause 16. Steerable tube according to clause 4 and 5, comprising a compensating device that is constructed and arranged for compensating a change in length of the intermediate layer in a longitudinal direction of the steerable tube upon twisting and/or untwisting the longitudinal elements.

Clause 17. Steerable tube according to clause 4 and 5, wherein the movable portions of the first and/or the third parts are constructed and arranged for compensating a change in length of the intermediate layer in a longitudinal direction of the steerable tube upon twisting and/or untwisting the longitudinal elements.

Clause 18. Endoscopic instrument comprising a steerable tube according to any one of the preceding clauses.

Clause 19. Endoscope comprising a steerable tube according to any one of the clauses 1-17.

Clause 20. Assembly comprising an endoscopic instrument according to clause 18 and an endoscope according to clause 19.

It will thus be seen that the present invention efficiently attains the objects set forth above, among those made apparent from the preceding description. Since certain changes can be made in the above constructions without departing from the scope of the invention, it is intended that all matter contained in the above description or shown in the accompanying drawings be interpreted as illustrative and not in a limiting sense.

It is also to be understood that the following claims are to cover all generic and specific features of the invention described herein, and all statements of scope of the invention which, as a matter of language, might be said to fall there between.

1. Steerable tube for endoscopic applications comprising a first, a second and a third part, wherein the first part comprises a first movable portion that is constructed and arranged for movably connecting the first part to a first end of the second part, wherein the third part comprises a second movable portion that is constructed and arranged for movably connecting the third part to a second end of the second part, wherein the steerable tube comprises longitudinal elements that are constructed and arranged such that a change in orientation of the first movable portion of the first part with respect to the second part results in a change in orientation of the second movable portion of the third part with respect to the second part, wherein the longitudinal elements are constructed and arranged such that they are twisted and/or that they are twistable.

2. Steerable tube according to claim 1, wherein a direction of the change in orientation of the second movable portion of the third part with respect to the second part is adjustable by selecting an angle of twist of the longitudinal elements.

3. Steerable tube according to claim 2, wherein the angle of twist of longitudinal elements is preset.

4. Steerable tube according to claim 2, wherein the angle of twist of longitudinal elements is adjustable.

5. Steerable tube according to claim 1, comprising an inner, an intermediate and an outer concentric layer, wherein the inner and outer layers are constructed and arranged for shielding the intermediate layer, wherein the intermediate layer comprises longitudinal elements, wherein the inner, intermediate and outer layers comprise movable portions in the first and third parts of the steerable tube, wherein movable portions of the first parts of the inner, intermediate and outer layers and movable portions of the third parts of these layers are located opposite to each other in a radial direction of the steerable tube.

6. Steerable tube according to claim 5, wherein the intermediate layer comprises longitudinal slits for forming the longitudinal elements.

7. Steerable tube according to claim 1, wherein a spacer element is provided between the longitudinal elements.

8. Steerable tube according to claim 1, wherein the first and third parts comprise a corresponding number of at least two independent movable portions, wherein changes in orientation of the first and a third independent movable portion of the first part with respect to the second part are transferable to respectively the second and a fourth independent movable portion of the third part via separate sets of longitudinal elements.

9. Steerable tube according to claim 5, wherein the first and third parts comprise a corresponding number of at least two independent movable portions, wherein changes in orientation of the first and a third independent movable portion of the first part with respect to the second part are transferable to respectively the second and a fourth independent movable portion of the third part via separate sets of longitudinal elements, wherein the intermediate layer comprises at least two concentric layers, wherein each concentric layer is constructed and arranged for comprising at least one separate set of longitudinal elements.

10. Steerable tube according to claim 9, wherein each separate set of longitudinal elements is arranged in a separate concentric layer of the intermediate layer.

11. Steerable tube according to claim 4, comprising an adjustment device that is constructed and arranged for adjusting the angle of twist of longitudinal elements.

12. Steerable tube according to claim 11, comprising a locking device that is constructed and arranged for maintaining a position of the adjustment device.

13. Steerable tube according to claim 4, comprising an inner, an intermediate and an outer concentric layer, wherein the inner and outer layers are constructed and arranged for shielding the intermediate layer, wherein the intermediate layer comprises longitudinal elements, wherein the inner, intermediate and outer layers comprise movable portions in the first and third parts of the steerable tube, wherein movable portions of the first parts of the inner, intermediate and outer layers and movable portions of the third parts of these layers are located opposite to each other in a radial direction of the steerable tube, wherein the steerable tube comprises a compensating device that is constructed and arranged for compensating a change in length of the intermediate layer in a longitudinal direction of the steerable tube upon twisting and/or untwisting the longitudinal elements.

14. Endoscopic instrument comprising a steerable tube according to claim 1.

15. Endoscope comprising a steerable tube according to claim 1.

16. Assembly comprising an endoscopic instrument according to claim 14.
